(19) United States
(12) Patent Application Publication
(10) Pub. No.: US 2012/0329826 A1
(43) Pub. Date: Dec. 27, 2012

(54) SUBSTITUTED-5-AMINOPYRROLO/PYRAZOLOPYRIDINES

(75) Inventors: An-Hu Li, Commack, NY (US); Ramesh C. Gupta, Port Jefferson Station, NY (US); Mark J. Mulvihill, Dix Hills, NY (US); Anand Narain Rai, Port Jefferson Station, NY (US); Jing Wang, Syosset, NY (US)

(73) Assignee: OSI Pharmaceuticals,. LLC

(21) Appl. No.: 13/581,619

(22) PCT Filed: Mar. 3, 2011
(86) PCT No.: PCT/US11/26984

§ 371 Date: Aug. 29, 2012

Related U.S. Application Data
(60) Provisional application No. 61/309,936, filed on Mar. 3, 2010.

(51) Int. Cl. C07D 471/02; A61P 35/04; A61K 31/437

(52) U.S. Cl. 514/300; 546/113

(57) ABSTRACT

Compounds of Formula I, as shown below and defined herein: pharmaceutically acceptable salts, synthesis, intermediates, formulations, and methods of disease treatment therewith, including cancers mediated at least in part by RON and/or MET.

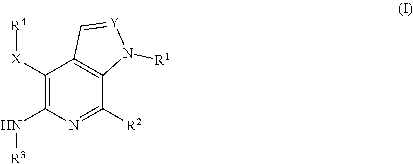

This application claims the benefit of prior U.S. Appl. No. 61/309,936 (filed Mar. 3, 2010), the contents of which are incorporated herein in their entirety by this reference.

### FIELD AND BACKGROUND

The present invention pertains at least in part to cancer treatment, certain chemical compounds, and methods of treating tumors and cancers with the compounds.

RON (recepteur d'origine nantais) is a receptor tyrosine kinase that is part of the MET proto-oncogene family. It is activated by binding to its natural ligand MSP and signals via the PI3K and MAPK pathways. RON can be deregulated in cancer by mechanisms such as over-expression of the receptor and/or the presence of constitutively active splice variants. Inhibition of RON has been shown to lead to a decrease in proliferation, induction of apoptosis and affects cell metastasis. RON overexpression is observed in a variety of human cancers and exhibit increased expression with progression of the disease.

MET (also known as c-Met or cMet) is a receptor tyrosine kinase that is a heterodimeric protein comprising of a 50 kDa α-subunit and a 145 kDa β-subunit (Maggiora et al., J. Cell Physiol., 173:183-186, 1997). It is activated by binding to its natural ligand HGF (hepatocyte growth factor, also known as scatter factor) and signals via the PI3K and MAPK pathways. MET can be deregulated in cancer by mechanisms such as autocrine/paracrine HGF activation, over-expression of the receptor, and/or the presence of activating mutations. Significant expression of MET has been observed in a variety of human tumors, such as colon, lung, prostate (including bone metastases), gastric, renal, HCC, ovarian, breast, ESCC, and melanoma (Maulik et al., Cytokine & Growth Factor Reviews 13:41-59, 2002). MET is also implicated in atherosclerosis and lung fibrosis. Inhibition of MET can cause a decrease in cell motility, proliferation and metastasis, as reviewed in, e.g., Chemical & Engineering News 2007, 85 (34), 15-23.

Elevated expression of c-MET has been detected in numerous cancers including lung, breast, colorectal, prostate, pancreatic, head and neck, gastric, hepatocellular, ovarian, renal, glioma, melanoma, and some sarcomas (See reviews Christensen, J., 2005; Comoglio, P., 2008). c-MET gene amplification and resulting overexpression has been reported in gastric and colorectal cancer (Smolen, G., 2005; Zeng Z., 2008). Taken together, the c-MET proto-oncogene has a role in human cancer and its over-expression correlates with poor prognosis. Abrogation of cMET function with small molecule inhibitors, anti-cMET antibodies or anti-HGF antibodies in preclinical xenograft model systems have shown impact when c-MET signaling serves as the main driver for proliferation and cell survival (Comoglio, P., 2008).

As human cancers progress to a more invasive, metastatic state, multiple signaling programs regulating cell survival and migration programs are observed depending on cell and tissue contexts (Gupta and Massague, 2006). Recent data highlight the transdifferentiation of epithelial cancer cells to a more mesenchymal-like state, a process resembling epithelial-mesenchymal transition (EMT); (Oft et al., 1996; Perl et al., 1998), to facilitate cell invasion and metastasis (Brabletz et al., 2005; Christofori, 2006). Through EMT-like transitions mesenchymal-like tumor cells are thought to gain migratory capacity at the expense of proliferative potential. A mesenchymal-epithelial transition (MET) has been postulated to regenerate a more proliferative state and allow macrometastases resembling the primary tumor to form at distant sites (Thiery, 2002). MET and RON kinases have been shown to play a role in the EMT process (Camp et al., 2007; Grotegut et al., 2006; Wang et al., 2004). It has been documented in vitro that RON and MET can form heterodimers and signal via such RON-MET dimers.

cMET and RON are known to interact and influence the activation of one another. Furthermore, co-expression of the two receptors, when compared to each receptor alone, is associated with the poorest clinical prognosis in bladder, CRC, and breast cancer patients. Since co-expression of RON and MET in cancer has been observed, such “cross-talk” may contribute to tumor growth.

US 2009/0197862, US 2009/0197864, and PCT/US09/65058 disclose kinase inhibitors. US 2009/0062273 discloses chemical compounds said to be Tie2 inhibitors.

There is a need for effective therapies for use in proliferative disease, including treatments for primary cancers, prevention of metastatic disease, and targeted therapies, including tyrosine kinase inhibitors, such as MET and/or RON inhibitors, dual inhibitors, including selective inhibitors, and for potent, orally bioavailable, and efficacious inhibitors, and inhibitors that maintain sensitivity of epithelial cells to epithelial cell directed therapies.

### SUMMARY

In some aspects, the present invention concerns compounds of Formula I, as shown

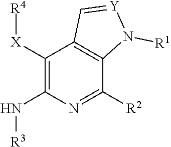

where variable substituents are defined herein. The invention includes the compounds and pharmaceutically acceptable salts thereof.

The invention includes the compounds and salts thereof, and their physical forms, preparation of the compounds, useful intermediates, and pharmaceutical compositions and formulations thereof.

In some aspects, compounds of the invention are useful as inhibitors of kinases, including at least one of the c-MET, ALK, and RON kinases.

In some aspects, compounds of the invention are useful in treating proliferative disease, particularly cancers, including cancers mediated by c-MET and/or RON and/or ALK, alone or in combination with other agents.

### DETAILED DESCRIPTION

### Compounds

In some aspects, the present invention concerns compounds of Formula I, as shown below and defined herein:

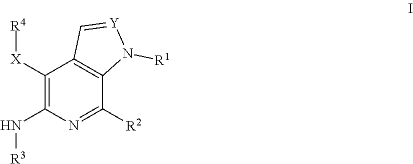

or a pharmaceutically acceptable salt thereof, wherein:

X is O, S(O)₀₋₂, or NR⁵; or X is absent;

Y is C—R⁶ or N;

R¹ is H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic, heteroarylC₀₋₁₂aliphatic, —OR⁷, —S(O)₀₋₂R⁸, —NR⁹R¹⁰, —SO₂NR⁹R¹⁰, —C(O)R^{a}, —C(O)NR⁹R¹⁰, —C(O)—C(O)NR⁹R¹⁰, —C(O)OR⁷, —C(O)—C(O)OR⁷, —OC(O)R^{b}, —NR⁹C(O)R^{a}, —NR⁹S(O)₂R^{a}, —(CR¹¹R¹²)ₙC(O)R^{a}, —(CR¹¹R¹²)ₙC(O)OR⁷, —(CR¹¹R¹²)ₙC(O)NR⁹R¹⁰, —(CR¹¹R¹²)ₙS(O)₂NR⁹R¹⁰, —(CR¹¹R¹²)ₙNR⁹R¹⁰, —(CR¹¹R¹²)ₙOR⁷, —(CR¹lR¹²)ₙS(O)₀₋₂R⁸, —NR¹³C(O)NR⁹R¹⁰, —NR¹³S(O)₂NR⁹R¹⁰ or —NR¹³S(O)NR⁹R¹⁰, any of which is optionally substituted with one or more independent G¹ substituents;

R² is H or halogen;

R³ is H or C₁₋₁₂aliphatic;

R⁴ is H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic, arylC₃₋₁₂cycloalkyl, arylC₃₋₁₂heterocycloalkyl, heteroarylC₀₋₁₂aliphatic, heteroarylC₃₋₁₂cycloalkyl or heteroarylC₃₋₁₂heterocycloalkyl, any of which is optionally substituted with one or more independent G² substituents;

or R⁴ is —(CR¹⁸R¹⁹)ₙA¹;

A¹ is aryl or heteroaryl optionally substituted by one or more independent G³;

R⁵ is H, C₁₋₁₂cycloalkylC₃₋₁₂aliphatic, C₃₋₁₂heterocycloalkylC₀₋₁₂aliphatic, arylC₀₋₁₂aliphatic, heteroarylC₀₋₁₂aliphatic, C₁₋₁₂aliphatic-S(O)₀₋₂—C₂₋₁₂aliphatic, (C₀₋₁₂aliphatic)(C₀₋₁₂aliphatic)N—C₂₋₁₂aliphatic, any of which is optionally substituted with one or more independent G⁴ substituents;

R⁶ is H, C₁₋₁₂cycloalkylC₃₋₁₂aliphatic, C₀₋₁₂heterocycloalkylC₃₋₁₂aliphatic, arylC₀₋₁₂aliphatic or heteroarylC₀₋₁₂aliphatic, any of which is optionally substituted with one or more independent G⁵ substituents, or R⁶ is halo, —CN, or —CF₃;

R⁷, R⁸, R⁹, R¹⁰, R¹¹, R¹², R¹³, R¹⁵, R¹⁶, R¹⁷, R¹⁸, R¹⁹, R^{a}, and R^{b} are each independently selected from H, C₁₋₁₂cycloalkylC₃₋₁₂aliphatic, C₀₋₁₂heterocycloalkylC₀₋₁₂aliphatic, arylC₀₋₁₂aliphatic or heteroarylC₀₋₁₂aliphatic;

R⁹ and R¹⁰, or R¹⁶ and R¹⁷ in NR⁹R¹⁰ and NR¹⁶R¹⁷, respectively, can be taken together with the nitrogen atom to which they are attached to form a 3-12 membered saturated or unsaturated ring, wherein said ring optionally includes one or more heteroatoms selected from O, N, or S(O)₀₋₂;

R¹¹ and R¹², or R¹⁸ and R¹⁹ in CR¹¹R¹² and CR¹⁸R¹⁹, respectively, can be taken together with the carbon atom to which they are attached to form a 3-12 membered saturated or unsaturated ring, wherein said ring optionally includes one or more heteroatoms selected from O, N, or S(O)₀₋₂;

G¹, G², G³, G⁴, and G⁵ are each independently selected from H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic, arylC₃₋₁₂cycloalkyl, arylC₃₋₁₂heterocycloalkyl, heteroarylC₀₋₁₂aliphatic, heteroarylC₃₋₁₂cycloalkyl or heteroarylC₃₋₁₂heterocycloalkyl, any of which is optionally substituted with one or more independent Q¹ substituents, or G¹, G², G³, G⁴, and G⁵ are each independently halo, —CN, —CF₃, —OCF₃, or —NO₂;

each Q¹ is independently selected from H, C₃₋₁₂cycloalkyl, C₃₋₁₂cycloalkyl, C₃₋₁₂heterocycloalkyl, aryl, heteroaryl, —C(O)—C(O)NR²⁰R²¹, C(O)—C(O)OR²², —OC(O)R^{c}, —NR²⁰C(O)R^{c}, —NR²⁰S(O)₂R²³, —(CR²⁴R²⁵)ₙC(O)R^{c}, —(CR²⁴R²⁵)ₙC(O)OR²², —(CR²⁴R²⁵)ₙC(O)NR²⁹R²¹, (CR²⁴R²⁵)ₙS(O)₂NR²⁰R²¹, (CR²⁴R²⁵)ₙNR²⁰R²¹, (CR²⁴R²⁵)ₙOR²², —(CR²⁴R²⁵)ₙC(O)NR²⁰R²¹, —(CR²⁴R²⁵)ₙS(O)S(O)₂NR²⁰R²¹, or —NR²⁶S(O)NR²⁰R²¹, any of which is optionally substituted with one or more independent Q² substituents or Q¹ is halo, —CN, —NO₂, oxo, —CF₃, or —OCF₃;

each Q² is independently selected from H, halo, —CN, —OH, —NH₂, —NO₂, oxo, —CF₃, —OCF₃, —CO₂H, —S(O)₀₋₂H, C₁₋₁₂aliphatic, C₃₋₁₂cycloalkyl, C₃₋₁₂heterocycloalkyl, aryl, heteroaryl, any of which is optionally substituted with one or more independent halo, —CN, —OH, —NH₂ or C₁₋₁₀alkyl which may be partially or fully halogenated, or —O—C₁₋₁₀alkyl which may be partially or fully halogenated;

R²⁰, R²¹, R²², R²³, R²⁴, R²⁵, R²⁶, and R^{c} are each independently selected from H, C₁₋₁₂aliphatic, arylC₀₋₁₂aliphatic, heteroarylC₀₋₁₂aliphatic, C₃₋₁₂cycloalkylC₀₋₁₂aliphatic, C₃₋₁₂heterocycloalkylC₀₋₁₂aliphatic, arylC₃₋₁₂cycloalkyl, heteroarylC₃₋₁₂cycloalkyl, C₃₋₁₂heterocycloalkylC₃₋₁₂cycloalkyl, C₃₋₁₂cycloalkylC₃₋₁₂cycloalkyl, C₁₋₁₂alkylC₃₋₁₂heterocycloalkyl, C₃₋₁₂heterocycloalkylC₃₋₁₂heterocycloalkyl, arylC₃₋₁₂heterocycloalkyl, or heteroaryl C₃₋₁₂heterocycloalkyl substituents;

R²⁰ and R²¹ in NR²OR²¹ can be taken together with the nitrogen atom to which they are attached to form a 3-12 membered saturated or unsaturated ring, wherein said ring optionally includes one or more heteroatoms selected from O, N, or S(O)₀₋₂;

R²⁴ and R²⁵ in CR²⁴R²⁵ can be taken together with the carbon atom to which they are attached to form a 3-12 membered saturated or unsaturated ring, wherein said ring optionally includes one or more heteroatoms selected from O, N, or S(O)₀₋₂; and

n=0-7.

In some aspects, there is provided a compound or salt of Formula I (as defined above), wherein (subgenus 1): X is O; Y is CH; R² is H or Cl; R³ is H; and R⁴ is —CH(CH₃)-A¹.

In some aspects, there is provided a compound or salt of Formula I (as defined above), or of subgenus 1 thereof, having the formula (subgenus 2):

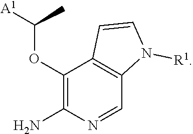

In some aspects, there is provided a compound or salt of Formula I (as defined above), or of subgenus 1 or 2 thereof, wherein (subgenus 3): A¹ is phenyl substituted by one or more independent halogen or methoxy optionally substituted by 1-3 fluorine atoms.

In some aspects, there is provided a compound or salt of Formula I (as defined above), or of subgenera 1-3 thereof, wherein (subgenus 4): A¹ is 2,6-dichloro-3-fluorophenyl. In some aspects, there is provided a compound or salt of Formula I (as defined above), or of subgenera 1-4 thereof, wherein (subgenus 5): R¹ is H, C₁₋₄aliphatic optionally substituted by ₅₋₆cyclic which is optionally substituted, —S(O)₀₋₂R⁸, or —C(O)OR⁷.

In some aspects, there is provided a compound or salt of Formula I (as defined above), or of subgenera 1-4 thereof, wherein (subgenus 6): R¹ is H or C₁₋₄aliphatic optionally substituted by ₅₋₆cyclic which is optionally substituted.

In some aspects, there is provided a compound or salt of Formula I (as defined above), or of subgenera 1-4 thereof, wherein (subgenus 7): R¹ is H or C₁₋₄aliphatic optionally substituted by ₅₋₆heterocyclic which is optionally substituted.

In some aspects, there is provided a compound or salt of Formula I (as defined above), having the formula (subgenus 8):

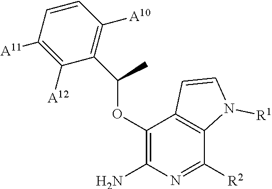

wherein:

A¹⁰ is halogen, methyl, or methoxy either of which is optionally substituted by 1-3 fluorine atoms;

A¹¹ and A¹² are independently halogen;

R¹ is H, C₁₋₄alkyl, —CH₂—CH₂-G¹, —CH₂—CH₂—NH-G¹, or —CH₂—CH₂—O-G¹;

R² is H; and

G¹ is ₄₋₆heterocycloalkyl optionally substituted by one or more substituted or unsubstituted oxo, aliphatic, carboxy, amido, sulfonamido, sulfone, sulfide, sulfoxide, or acyl.

In some aspects, there is provided a compound or salt of Formula I (as defined above), having the formula (subgenus 9):

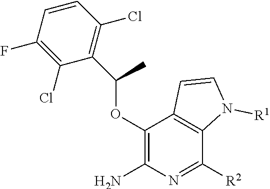

wherein:

R¹ is H or C₁₋₃aliphatic optionally substituted by ₅₋₆cyclic; and

R² is H or halogen.

In some aspects, there is provided a compound or salt of Formula I (as defined above), wherein (subgenus 10):

X is absent;

Y is CH;

R¹ is H or C₁₋₄alkyl optionally substituted by ₅₋₆cyclic or aryl which is optionally substituted;

R² is H or Cl;

R³ is H; and

R⁴ is benzofuran-2-yl which can be substituted by 1-3 independent halogen, hydroxy, or —OC₀₋₃aliphatic optionally substituted by 1-3 halogen atoms.

In some aspects, there is provided a compound or salt of subgenus 10, wherein (subgenus 11): R² is H.

In some aspects, there is provided a compound or salt of Formula I (as defined above), having the formula (subgenus 12):

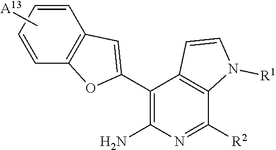

wherein:

A¹³ is H, halogen, methyl, or methoxy either of which is optionally substituted by 1-3 fluorine atoms;

R¹ is H, C₁₋₄alkyl, —CH₂—CH₂-G¹, —CH₂—CH₂—NH-G¹, or —CH₂—CH₂—O-G¹;

R² is H; and

G¹ is ₄₋₆heterocycloalkyl optionally substituted by one or more substituted or unsubstituted oxo, aliphatic, carboxy, amido, sulfonamido, sulfone, sulfide, sulfoxide, or acyl.

In some embodiments, the compound is selected from any one of the examples herein.

Each variable definition above includes any subset thereof and the compounds of Formula I include any combination of such variables or variable subsets.

US 2009/0197864 is incorporated by reference herein for the purpose of its descriptions of various substituents (R²) corresponding to R¹ herein and its R⁴ corresponding to R⁴ herein.

The invention includes a compound of Formula I or a pharmaceutically acceptable salt thereof, which is sufficiently orally bioavailable for effective oral human administration.

The invention includes a compound of Formula I or a pharmaceutically acceptable salt thereof, which has a suitable therapeutic window for effective human administration, oral or otherwise.

In some aspects, the invention includes any of the compound examples herein and pharmaceutically acceptable salts thereof.

The invention includes the compounds and salts thereof, and their physical forms, preparation of the compounds, useful intermediates, and pharmaceutical compositions and formulations thereof.

The compounds of the invention and term “compound” in the claims include any pharmaceutically acceptable salts or solvates, and any amorphous or crystal forms, or tautomers, whether or not specifically recited in context.

The invention includes the isomers of the compounds. Compounds may have one or more asymmetric carbon atoms can exist as two or more stereoisomers. Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. A single compound may exhibit more than one type of isomerism.

The present invention includes any stereoisomers, even if not specifically shown, individually as well as mixtures, geometric isomers, and pharmaceutically acceptable salts thereof. Where a compound or stereocenter is described or shown without definitive stereochemistry, it is to be taken to embrace all possible individual isomers, configurations, and mixtures thereof. Thus, a material sample containing a mixture of stereoisomers would be embraced by a recitation of either of the stereoisomers or a recitation without definitive stereochemistry. Also contemplated are any cis/trans isomers or tautomers of the compounds described.

Included within the scope of the invention are all stereoisomers, geometric isomers and tautomeric forms of the inventive compounds, including compounds exhibiting more than one type of isomerism, and mixtures of one or more thereof.

When a tautomer of the compound of Formula (I) exists, the compound of formula (I) of the present invention includes any possible tautomers and pharmaceutically acceptable salts thereof, and mixtures thereof, except where specifically stated otherwise.

The compounds of the invention are not limited to those containing all of their atoms in their natural isotopic abundance. The present invention includes compounds wherein one or more hydrogen, carbon or other atoms are replaced by different isotopes thereof. Such compounds can be useful as research and diagnostic tools in metabolism pharmacokinetic studies and in binding assays. A recitation of a compound or an atom within a compound includes isotopologs, i.e., species wherein an atom or compound varies only with respect to isotopic enrichment and/or in the position of isotopic enrichment. For nonlimiting example, in some cases it may be desirable to enrich one or more hydrogen atoms with deuterium (D) or to enrich carbon with ¹³C. Other examples of isotopes suitable for inclusion in the compounds of the invention include isotopes of hydrogen, chlorine, fluorine, iodine, nitrogen, oxygen, phosphorus, and sulfur. Certain isotopically-labeled compounds of the invention may be useful in drug and/or substrate tissue distribution studies. Substitution with heavier isotopes such as deuterium may afford certain therapeutic advantages resulting from greater metabolic stability, for example, increased in vivo half-life or reduced dosage requirements, and hence may be preferred in some circumstances. Substitution with positron emitting isotopes may be useful in Positron Emission Topography (PET) studies for examining substrate receptor occupancy.

Further, the compounds may be amorphous or may exist or be prepared in various crystal forms or polymorphs, including solvates and hydrates. The invention includes any such forms provided herein, at any purity level. A recitation of a compound per se means the compound regardless of any unspecified stereochemistry, physical form and whether or not associated with solvent or water.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g., D₂O, d₆-acetone, d6-DMSO.

Also included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized.

The invention includes prodrugs of compounds of the invention which may, when administered to a patient, be converted into the inventive compounds, for example, by hydrolytic cleavage. Prodrugs in accordance with the invention can, for example, be produced by replacing appropriate functionalities present in the inventive compounds with certain moieties known to those skilled in the art as ‘pro-moieties’ as known in the art. Particularly favored derivatives and prodrugs of the invention are those that increase the bioavailability of the compounds when such compounds are administered to a patient, enhance delivery of the parent compound to a given biological compartment, increase solubility to allow administration by injection, alter metabolism or alter rate of excretion.

A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Compounds that are basic are capable of forming a wide variety of salts with various inorganic and organic acids. The acids that can be used to prepare pharmaceutically acceptable acid addition salts of such basic compounds are those that form acceptable acid addition salts. When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Other salts are aspartate, besylate, bicarbonate/carbonate, bisulphate/sulfate, borate, camsylate, edisylate, gluceptate, glucuronate, hexafluorophosphate, hibenzate, hydrobromide/bromide, hydroiodide/iodide, malonate, methylsulfate, naphthylate, 2-napsylate, nicotinate, orotate, oxalate, palmitate, phosphate/hydrogen, phosphate/dihydrogen, phosphate, saccharate, stearate, tartrate, tosylate, and trifluoroacetate.

When the compound of the present invention is acidic, its corresponding salt can be conveniently prepared from pharmaceutically acceptable bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic bases include salts of primary, secondary, and tertiary amines, as well as cyclic amines and substituted amines such as naturally occurring and synthesized substituted amines. Other pharmaceutically acceptable organic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Other examples include benzathine, diolamine, glycine, meglumine, and olamine.

### General Synthesis

The invention includes the intermediates, examples, and synthetic methods described herein.

The compounds of the Formula I may be prepared by the methods described below, together with synthetic methods known in the art of organic chemistry, or modifications and derivatizations that are familiar to those of ordinary skill in the art. In particular, reference is made to the general chemistries described in US 2009/0197864, pp. 26-29. The starting materials used herein are commercially available or may be prepared by routine methods known in the art [such as those methods disclosed in standard reference books such as the Compendium of Organic Synthetic Methods, Vol. I-VI (Wiley-Interscience); or the Comprehensive Organic Transformations, by R. C. Larock (Wiley-Interscience)]. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed hereinbelow and the general skill in the art. Unless otherwise indicated, the substituents in the Schemes are defined as above. Isolation and purification of the products is accomplished by standard procedures, which are known to a chemist of ordinary skill.

When a general or exemplary synthetic procedure is referred to, one skilled in the art can readily determine the appropriate reagents, if not indicated, extrapolating from the general or exemplary procedures. Some of the general procedures are given as examples for preparing specific compounds. One skilled in the art can readily adapt such procedures to the synthesis of other compounds. Representation of an unsubstituted position in structures shown or referred to in the general procedures is for convenience and does not preclude substitution as described elsewhere herein. For specific groups that can be present, either as R groups in the general procedures or as optional substituents not shown, refer to the descriptions in the remainder of this document, including the claims, summary and detailed description.

Compounds of the invention can be prepared according to Scheme 1, wherein the variables shown have the definitions of the description herein unless otherwise indicated.

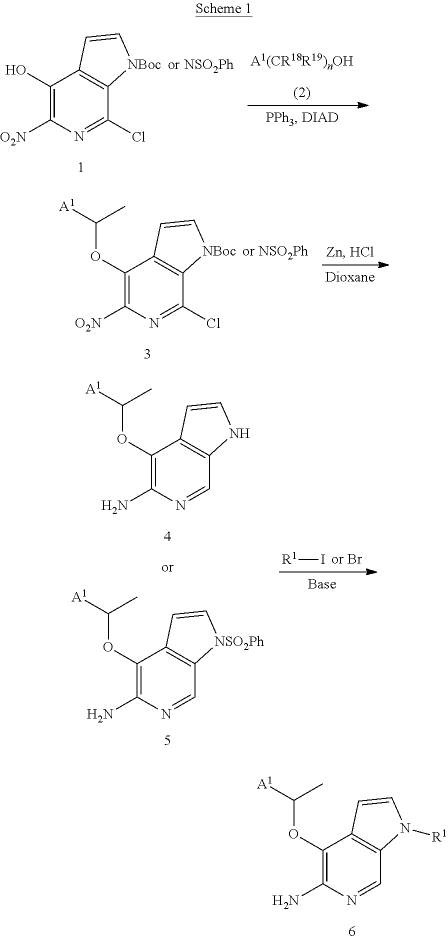

Compounds 6 (of Formula I) can be prepared, for example, from Compound 1 (Intermediate C or H). Compound 3 can be prepared under Mitsunobu conditions (such as in the manner of Intermediate D or I (below)) by reacting a solution (e.g., THF) of Compound I with an appropriate alcohol (about 1 eq) in the presence of Ph₃P (about 1.5 eq) and DIAD (about 1.5 eq) at rt for about 2 h, followed by workup. Alternatively, Compound 1 can be alkylated with an analogous reagent having an appropriate leaving group in the presence of base.

The 7-Cl and 5-nitro groups of Compound 3 can be reduced in various ways to 4 or 5. For example, to a solution of the starting material in dioxane with zinc dust (e.g., about 20 eq) can slowly be added conc. aq. HCl and reacted at room temperature, followed by workup (See, e.g., Example 1). In this manner, a protecting Boc group is also removed. Alternatively, Compound 3 can be partially reduced in the manner of Intermediate E (below).

Following deprotection, if necessary, Compound 6 can be obtained such as by reaction with an appropriate alkyl halide (multiple equivalents) in the presence of Cs₂CO₃, such as in the manner of Example 2 (below). Alternative conditions include using leaving groups such as, but not limited to, mesylate, tosylate, or triflate, under typical alkylation conditions such as, but not limited to, K₂CO₃ or Cs₂CO₃ as base in a polar aprotic solvent such as DMF.

The 5-position amine of Compound 6 can be derivatized (introducing R³) by methods such as reductive amination.

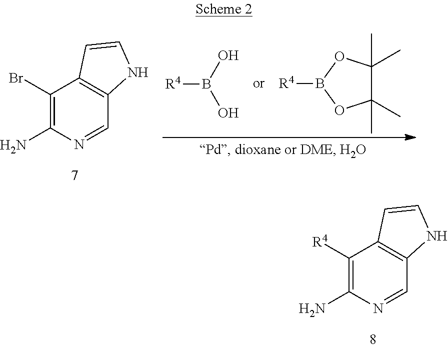

The general methods to convert the Br group to R⁴ to obtain Compound 8 include Suzuki coupling with (het)arylboronic acids or boronates, with vinylboronates, alkylboronates, or 9-BBN-derived alkylboranes; Stille coupling with (het)arylstannanes or vinylstannanes; Negishi coupling with dialkylzinc reagents, alkylzinc halides, or (het)arylzinc halides; Sonogashira coupling with terminal alkynes; Cu- or Pd-mediated cyanations; Cu-mediated trifluoromethylations; and Pd-mediated carbonylations. Thus, Compounds 8 can be prepared by reacting Compounds 7 with an appropriate boronic acid or boronic acid ester as in Example 7 (below).

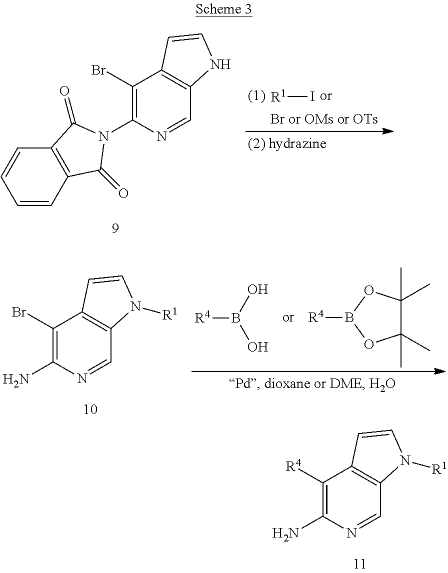

Compound 10 can be prepared in a manner analogous to Compound 6 (Scheme 1, above), followed by deprotection of the amine. Compound 11 can be prepared from 10 in a manner analogous to Scheme 2.

### Preparations

Intermediate A:

### tert-Butyl 4-bromo-7-chloropyrrolo[2,3-c]pyridine-1-carboxamide

A solution of 4-bromo-7-chloropyrrolo[2,3-c]pyridine (1.06 g, 4.5 mmol) (prepared from 2-chloro-3-nitro-5-bromopyridine according to Z. Zhang et al JOC 2002, 67, 2345), DMAP (585 mg, 4.73 mmol) and (Boc)₂O (950 mg, 4.73 mmol) in THF (20 mL) was stirred at rt for 1 h. The reaction mixture was concentrated, diluted with ethyl acetate (20 mL) and washed with 5% aq. NaHSO₄ (3×20 mL). The organic phase was washed with water, brine, dried over Na₂SO₄, filtered and concentrated in vacuo to give 1.1 g (76%) of the title compound. ¹H NMR (300 MHz, CDCl₃): δ 1.68 (s, 9H), 6.68 (d, J=3.6 Hz, 1H), 7.77 (d, J=3.9 Hz, 1H), 8.27 (s, 1H).

Intermediate B:

### tert-Butyl 4-hydroxyl-7-chloropyrrolo[2,3-c]pyridine-1-carboxamide

A solution of Pd₂(dba)₃ (226 mg, 0.24 mmol), tricyclohexylphosphine (340 mg, 1.21 mmol) in dioxane (50 mL) was passed through nitrogen for 30 min. Bis(pinacolato)diboron (4.2 g, 16.8 mmol), tert-butyl 4-bromo-7-chloropyrrolo[2,3-c]pyridine-1-carboxamide (4 g, 12.1 mmol) and KOAc (1.85 g, 19.3 mmol) were added. The reaction mixture was heated to 80° C. for 12 h. The reaction mixture was filtered and concentrated to give an oil, which was dissolved in dichloromethane (20 mL) and cooled to 0° C. 30% H₂O₂ (14 mL) was added and the resulting mixture was stirred for 4 h. The reaction was washed with water, dried over Na₂SO₄ and concentrated in vacuo to give a crude mixture, which was purified by silica gel column chromatography (30% EtOAc/hexanes) to afford 1.3 g (41%) of the desired product. ¹H NMR (300 MHz, DMSO-d₆): δ 1.60 (s, 9H), 6.82 (d, J=3.6 Hz, 1H), 7.70 (s, 1H), 7.78 (d. J=3.6 Hz, 1H), 10.45 (s, 1H).

Intermediate C:

### tert-Butyl 4-hydroxyl-5-nitro-7-chloropyrrolo[2,3-c]pyridine-1-carboxamide

A suspension of tert-butyl 4-hydroxyl-7-chloropyrrolo[2,3-c]pyridine-1-carboxamide (200 mg, 0.74 mmol), Ni(NO₃)₆ (282 mg, 0.98 mmol) and pTSA (28.5, 0.15 mmol) were heated to reflux for 1 h. The reaction mixture was concentrated and the crude reaction mixture was purified by silica gel column chromatography (DCM) to give 138 mg (58%) of the desired product. ¹H NMR (300 MHz, CDCl₃): δ 1.69 (s, 9H), 7.02 (d, J=3.6 Hz, 1H), 7.74 (d, J=3.6 Hz, 1H), 10.89 (s, 1H).

Intermediate D:

### tert-Butyl 7-chloro-5-nitro-4-[(R)-1-(2,6-dichloro-3-fluorophenyl)ethoxy]pyrrolo[2,3-c]pyridine-1-carboxamide

To a solution of tert-butyl 4-hydroxyl-5-nitro-7-chloropyrrolo[2,3-c]pyridine-1-carboxamide (75 mg, 0.24 mmol), Ph₃P (94 mg, 0.36 mmol) and (S)-1-(2,6-dichloro-3-fluorophenyl)ethanol (55 mg, 0.26 mmol, prepared according to literature procedures: WO2006/021881A2) was added DIAD (0.07 mL, 0.36 mmol). The reaction mixture was stirred at rt for 2 h. The resulting mixture was concentrated and the residue was purified by column chromatography (SiO₂, 20% EtOAc/hexanes) to give 67 mg (56%) of the title compound. ¹H NMR (300 MHz, CDCl₃): δ 1.67 (s, 9H), 1.91 (d, J=5.1 Hz, 3H), 6.11 (q, J=5.1 Hz, 1H), 6.69 (d, J=2.4 Hz, 1H), 7.61-7.11 (m, 1H), 7.30-7.32 (m, 1H), 7.69 (d, J=2.4 Hz, 1H).

Intermediate E:

### tert-Butyl 5-Amino-7-chloro-4-[(R)-1-(2,6-dichloro-3-fluorophenyl)ethoxy]pyrrolo[2,3-c]pyridine-1-carboxamide

To a solution of tert-butyl 7-chloro-5-nitro-4-[(R)-1-(2,6-dichloro-3-fluorophenyl)ethoxy]pyrrolo[2,3-c]pyridine-1-carboxamide (100 mg, 0.2 mmol) in acetic acid (5 mL) was added Zn dust (258 mg, 3.97 mmol). The resulting mixture was stirred for 16 h at room temperature. The reaction mixture was concentrated to dryness (bath temperature <40° C.). To the residue, aq. 10% sodium bicarbonate (10 mL) was added and the mixture was extracted with ethyl acetate (2×10 mL). The combined organic phases were dried (Na₂SO₄) and concentrated to give 35 mg of title compound (yield: 35%). ¹H NMR (300 MHz, CDCl₃): δ 1.65 (s, 9H), 1.80 (d, J=5 Hz, 3H), 4.59 (bs, 2H), 6.02 (q, J=5 Hz, 1H), 6.4 (d. J=2.4 Hz, 1H), 7.1 (m, 1H), 7.31 (m, 1H), 7.58 (d, J=2.4 Hz, 1H).

Intermediate F:

### 1-Benzenesulfonyl-4-bromo-7-chloro-1H-pyrrolo[2,3-c]pyridine

To a cold (−5° C.) suspension of NaH (6.9 g, 173.1 mmol) in THF (125 mL) was added a solution of 4-bromo-7-chloro-1H-pyrrolo[2,3-c]pyridine (20 g, 86.6 mmol) in THF (200 mL) over a period of 1h. The reaction mixture was stirred at −5 to 10° C. for 30 min. A solution of PhSO₂Cl (16.5 mL, 129 mmol) in THF (50 mL) was added over a period of 20 min. After stirred for another hour, the reaction mixture was quenched with 10% aq. sodium hydrogen phosphate (90 mmol). The reaction mixture was transferred to a separatory funnel, diluted with ethyl acetate (200 mL) and collected the organic layer. The organic layer was washed with 10% aq. sodium carbonate (100 mL) followed by water and brine, treated with charcoal, filtered, concentrated to dryness. The residue was triturated with diisopropyl ether to gave 26 g (81%) of the target compound after drying.

Intermediate G:

### 1-Benzenesulfonyl-7-chloro-1H-pyrrolo[2,3-c]pyridin-4-ol

To a degassed solution of Pd₂(dba)₂ (303 mg, 0.32 mmol) and tricyclohexylphsophine (454 mg, 1.62 mmol) in dioxane (70 mL) was added bis(pinacolato)diboron (5.68 g, 22.6 mmol), 1-benzenesulfonyl-4-bromo-7-chloro-1H-pyrrolo[2,3-c]pyridine (6.0 g, 16.2 mmol) and KOAc (2.5 g, 25.9 mmol). The reaction mixture was stirred for 12 h at 80° C. After cooled to room temperature, the mixture was filtered and concentrated to give crude residue, which was dissolve in dichloromethane (20 mL) and cooled to 0° C. To this solution was added 30° A H₂O₂ (14 mL) and the reaction mixture was stirred for 4 h. The organic layer was washed with water, dried over Na₂SO₄ and concentrated to give a residue, which was purified by column chromatography (SiO₂, 30% EtOAc/hexanes) to afford 1.5 g (31%) of the desired product. ¹H NMR (300 MHz, CDCl₃): δ 6.86-6.89 (d, J=3.3 Hz, 1H), 7.52-7.59 (m, 2H), 7.62-7.68 (m, 2H), 7.94-7.99 (m, 2H), 8.70 (s, 1H).

Intermediate H:

### 1-Benzenesulfonyl-7-chloro-5-nitro-1H-pyrrolo[2,3-c]pyridin-4-ol

A suspension of 1-benzenesulfonyl-7-chloro-1H-pyrrolo[2,3-c]pyridin-4-ol (870 mg, 2.82 mmol), Ni(NO₃)₂.6H₂O (1 g, 3.3 mmol) and pTSA (106 mg, 0.56 mmol) in acetone (30 mL) was heated to reflux for 2 h. The reaction mixture was concentrated and the crude reaction mixture was adsorbed on silica, which was purified by column chromatography (SiO₂, DCM) to give 550 mg (55%) of target product. ¹H NMR (300 MHz, CDCl₃): δ 7.20 (d, J=3.2 Hz, 1H), 7.60-7.71 (m, 2H), 7.73-7.78 (m, 2H), 7.90-7.95 (m, 2H), 10.90 (s, 1H).

Intermediate I:

### 1-Benzenesulfonyl-7-chloro-4-[(R)-1-(2,6-dichloro-3-fluorophenyl)ethoxy]-5-nitro-1H-pyrrolo[2,3-c]pyridine

To a cold (0° C.) solution of 1-benzenesulfonyl-7-chloro-5-nitro-1H-pyrrolo[2,3-c]pyridin-4-ol (350 mg, 1 mmol), Ph₃P (394 mg, 1.5 mmol) and (S)-1-(2,6-dichloro-3-fluorophenyl)ethanol (313 mg, 1.5 mmol) in THF (10 mL) was added DIAD (0.29 mL, 1.5 mmol). The reaction mixture was stirred at room temperature for 2 h, concentrated and purified by column chromatography (SiO₂, 20% EtOAc/hexanes) to give 280 mg (51%) of the title compound. ¹H NMR (300 MHz, CDCl₃): δ 1.60 (d, J=6.9 Hz, 3H), 5.84 (q, J=6.9 Hz, 1H), 6.57 (d, J=3.3 Hz, 1H), 6.82-6.88 (m, 1H), 7.04-7.08 (m, 1H), 7.26-7.32 (m, 2H), 7.40-7.42 (m, 1H), 7.54-7.58 (m, 2H), 7.83 (d, J=3.3 Hz, 1H).

Intermediate J:

### N′-[3-Bromo-4-((E)-2-dimethylaminovinyl)-5-nitropyridin-2-yl]-N,N-dimethylformamidine

A solution of 3-bromo-4-methyl-5-nitropyridin-2-ylamine (8 g, 34.4 mmol) and N,N-dimethylformamide dimethyl acetal (30 mL) in DMF (20 mL) was stirred at 110° C. for 12 h. The reaction mixture was concentrated to remove most of the N,N-dimethylformamide dimethyl acetal and the rest of the solution was added to diisopropyl ether with rapid stirring. After 1 h, the solid was filtered to give 10 g of the target product (84%) as a red crystalline solid.

Intermediate K:

### 4-Bromo-1H-pyrrolo[2,3-c]pyridin-5-ylamine

A solution of N′-[3-bromo-4-((E)-2-dimethylaminovinyl)-5-nitropyridin-2-yl]-N,N-dimethylformamidine (3.8 g, 11.1 mmol) in THF-EtOAc (200 mL, 1:1) was hydrogenated in the presence of Palladium on charcoal (380 mg, 10% by weight) at 1 atmospheric pressure to give N′-(4-bromo-1H-pyrrolo[2,3-c]pyridin-5-yl)-N,N-dimethylformamidine intermediate. The resulting mixture was filtered, concentrated and the residue was taken into a mixture of 10% aq. Na₂CO₃ (20 mL) and dioxane (20 mL), and heated under reflux for 12 h. The mixture was concentrated to dryness, and to the residue was added water (20 mL). The solid materials were collected by filtration, and dried to give 2.3 g (85%) of the title compound as a light brown solid. ¹H NMR (300 MHz, CDCl₃): δ 5.20 (bs, 2H), 6.2 (d, J=3 Hz, 1H), 7.6 (d, J=3 Hz, 1H), 8.2 (s, 1H), 11.4 (bs, 1H).

Intermediate L:

### 2-(4-Bromo-1H-pyrrolo[2,3-c]pyridin-5-yl)isoindole-1,3-dione

A mixture of 4-bromo-1H-pyrrolo[2,3-c]pyridin-5-ylamine (200 mg, 0.94 mmol) and phthalic anhydride (837 mg, 5.66 mmol) in acetic acid (4 mL) was heated under reflux for 12 h. The reaction mixture was concentrated and the residue was stirred with 10% aq. sodium carbonate (20 mL) for 2 h to remove the excess of phthalic anhydride. The solid was filtered and dried to give 250 mg (73%) of the desired product. ¹H NMR (300 MHz, CDCl₃): δ 6.50 (m, 1H), 7.40 (m, 1H), 7.67 (m, 2H), 7.81-7.84 (m, 2H), 8.59 (s, 1H), 11.40 (bs, 1H).

Intermediate M:

### 1-Benzyl-4-bromo-1H-pyrrolo[2,3-c]pyridin-5-ylamine

A mixture of 2-(4-bromo-1H-pyrrolo[2,3-c]pyridin-5-yl)isoindole-1,3-dione (120 mg, 0.35 mmol), benzyl bromide (0.05 mL, 0.42 mmol) and cesium carbonate (170 mg, 0.52 mmol) in DMF (3 mL) was stirred at 80° C. for 2 h. TLC indicated the reaction was over, hydrazine hydrate (0.05 mL, 1.14 mmol) was then added to the hot solution and stirring was continued for another 30 min. The reaction mixture was cooled to room temperature and diluted with ethyl acetate (10 mL). The organic layer was separated, washed with water (3×5 mL), concentrated, and the residue was triturated with heptane to give 78 mg (74%) of 1-benzyl-4-bromo-1H-pyrrolo[2,3-c]pyridin-5-ylamine.

Intermediate N:

### tert-Butyl 4-[2-(5-amino-4-bromopyrrolo[2,3-c]pyridin-1-yl)ethyl]piperidine-1-carboxamide

A mixture of 2-(4-bromo-1H-pyrrolo[2,3-c]pyridin-5-yl)isoindole-1,3-dione (100 mg, 0.29 mmol), tert-butyl 4-(2-methanesulfonyloxyethyl)piperidine-1-carboxamide (104 mg, 0.34 mmol) and cesium carbonate (137 mg, 0.42 mmol) in DMF was stirred at 80° C. for 12 h. TLC indicated the reaction was over, hydrazine hydrate (0.05 mL, 1.14 mmol) was then added to the hot solution and stirring was continued for another 30 min. The reaction mixture was cooled to room temperature and diluted with ethyl acetate (10 mL). The organic layer was separated, washed with water (3×5 mL), and concentrated give 90 mg (73%) of the desired product.

### EXAMPLES

### Example 1

### 4-[(R)-1-(2,6-Dichloro-3-fluorophenyl)ethoxy]-1H-pyrrolo[2,3-c]pyridin-5-ylamine

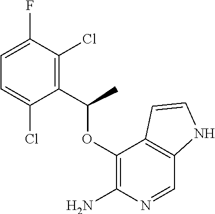

To a suspension of tert-butyl 5-Amino-7-chloro-4-[(R)-1-(2,6-dichloro-3-fluorophenyl)ethoxy]pyrrolo[2,3-c]pyridine-1-carboxamide (275 mg, 0.39 mmol) and Zn (1.03 g, 15.8 mmol) in dioxane was added conc. aq. HCl (1.35 mL, 15.8 mmol) over a period of 30 min. After stirred for another hour, the reaction mixture was basified with sodium bicarbonate and filtered. The filtrate was concentrated and the residue was taken into ethyl acetate (20 mL) and water (20 mL). The organic phase was separated, dried over Na₂SO₄ and concentrated to give 75 mg (55%) of the desired product. The crude product was used in next steps without purification. ¹H NMR (300 MHz, CDCl₃): δ 1.80 (d, J=6.9 Hz, 3H), 4.40 (bs, 2H), 6.22 (q, J=6.9 Hz, 1H), 6.40 (d, J=3.9 Hz, 1H), 7.04-7.10 (m, 2H), 7.29-7.33 (m, 1H), 8.08 (s and bs, 2H).

### Example 2

### 4-[(R)-1-(2,6-Dichloro-3-fluorophenyl)ethoxy]-1-methyl-1H-pyrrolo[2,3-c]pyridin-5-ylamine

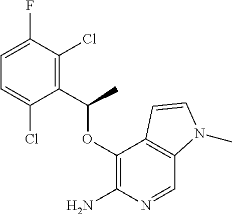

A mixture of 4-[(R)-1-(2,6-dichloro-3-fluorophenyl)ethoxy]-1H-pyrrolo[2,3-c]pyridin-5-ylamine (25 mg, 0.074 mmol), MeI (0.03 mL, 0.44 mmol) Cs₂CO₃ (72 mg, 0.22 mmol) in DMF (1 mL) was stirred at 40° C. for 4 h. The reaction mixture was taken into ethyl acetate (10 mL) and washed with water (5×10 mL). The organic layer was dried over Na₂SO₄, filtered, concentrated in vacuo to give 7 mg (26%) of the title compound. ¹H NMR (300 MHz, CDCl₃): δ 1.92 (d, J=6.9 Hz, 3H), 4.18 (s, 3H), 6.15 (q, J=6.9 Hz, 1H), 7.04-7.10 (m, 1H), 7.18 (d, J=3.3 Hz, 1H), 7.26-7.27 (m, 1H).

### Example 3

### 4-[(R)-1-(2,6-Dichloro-3-fluorophenyl)ethoxy]-1-ethyl-1H-pyrrolo[2,3-c]pyridin-5-ylamine

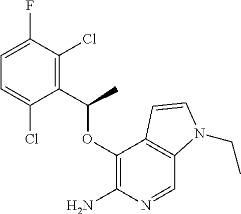

A mixture of 4-[(R)-1-(2,6-dichloro-3-fluorophenyl)ethoxy]-1H-pyrrolo[2,3-c]pyridin-5-ylamine (25 mg, 0.074 mmol), Etl (0.04 mL, 0.44 mmol) and Cs₂CO₃ (72 mg, 0.22 mmol) in DMF (1 mL) was stirred at 40° C. for 4 h. The resulting reaction mixture was taken into ethyl acetate (10 mL) and washed with water (5×10 mL). The organic layer was dried over Na₂SO₄, filtered, and concentrated in vacuo to give 12 mg (44%) of the title compound. ¹H NMR (300 MHz, CDCl₃): δ 1.48 (t, J=7.5 Hz, 3H), 1.80 (d, J=6.9 Hz, 3H), 4.14 (q, J=7.5 Hz, 2H), 4.40 (bs, 2H), 6.22 (q, J=6.9 Hz, 1H), 6.30 (d, J=3.9 Hz, 1H), 7.04-7.10 (m, 2H), 7.29-7.33 (m, 1H), 8.08 (s, 1H).

### Example 4

### tert-Butyl 4-(2-{5-amino-4-[(R)-1-(2,6-dichloro-3-fluorophenyl)ethoxy]pyrrolo[2,3-c]pyridin-1-yl}ethyl)piperidine-1-carboxamide

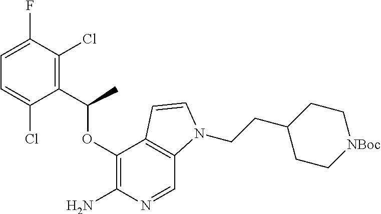

A mixture of 4-[(R)-1-(2,6-dichloro-3-fluorophenyl)ethoxy]-1H-pyrrolo[2,3-c]pyridin-5-ylamine (25 mg, 0.074 mmol), tert-butyl 4-(2-methanesulfonyloxyethyl)piperidine-1-carboxamide (135 mg, 0.44 mmol) and Cs₂CO₃ (72 mg, 0.22 mmol) in DMF (1 mL) was stirred at 60° C. for 4 h. The resulting reaction mixture was taken into ethyl acetate (10 mL) and washed with water (5×10 mL). The organic layer was dried over Na₂SO₄, filtered, and concentrated in vacuo to give crude residue which was purified by column chromatography (SiO₂, EtOAc/hexanes, 4/6) to afford 10 mg (25%) of the desired product. ¹H NMR (300 MHz, CDCl₃): δ 1.13-1.16 (m, 2H), 1.44 (s, 9H), 1.76-1.81 (m, 7H), 2.63 (m, 2H), 4.10-4.14 (m, 5H), 4.2 (bs, 2H), 6.21 (q, J=5.1 Hz, 1H), 6.30 (d, J=2.4 Hz, 1H), 7.05-7.09 (m, 2H), 7.29-7.32 (m 1H), 8.04 (s, 1H).

### Example 5

### 4-[(R)-1-(2,6-Dichloro-3-fluorophenyl)ethoxy]-1-(2-piperidin-4-ylethyl)1H-pyrrolo[2,3-c]pyridin-5-ylamine bis-hydrochloride

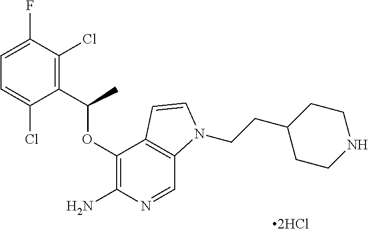

To a solution of Boc-derivative tert-butyl 4-(2-{5-amino-4-[(R)-1-(2,6-dichloro-3-fluorophenyl)ethoxy]pyrrolo[2,3-c]pyridin-1-yl}ethyl)piperidine-1-carboxamide in dichloromethane (1 mL) was added a drop of 4M HCl in dioxane. The reaction mixture was stirred for 12 h at room temperature. The reaction mixture was concentrated to dryness to give 4-[(R)-1-(2,6-dichloro-3-fluorophenyl)ethoxy]-1-(2-piperidin-4-ylethyl)1H-pyrrolo[2,3-c]pyridin-5-ylamine bis-hydrochloride as a white solid.

### Example 6

### 1-Benzenesulfonyl-4-[(R)-1-(2,6-dichloro-3-fluorophenyl)ethoxy]-1H-pyrrolo[2,3-c]pyridin-5-ylamine

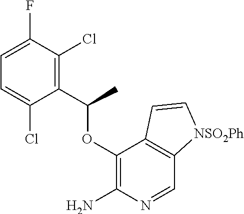

To a suspension of 1-benzenesulfonyl-7-chloro-4-[(R)-1-(2,6-dichloro-3-fluorophenyl)ethoxy]-5-nitro-1H-pyrrolo[2,3-c]pyridine (Intermediate I, 100 mg, 0.18 mmol) and Zn dust (500 g, 7.69 mmol) in dioxane (50 mL) was added conc. HCl (0.5 mL, 6 mmol) over a period of 30 min. The resulting mixture was stirred for another hour, and then diluted with ethyl acetate (20 mL), basified with sodium bicarbonate and filtered to remove unreacted zinc dust. The organic layer was separated, dried over Na₂SO₄ and concentrated to give 41 mg (47%) of the title compound. ¹H NMR (300 MHz, CDCl₃): δ 1.78 (d, J=6.9 Hz, 3H), 4.54 (bs, 2H), 5.97 (q, J=6.9 Hz, 1H), 6.49 (dd, J=0.9, 3.6 Hz, 1H), 7.06 (dd, J=6.9, 8.7 Hz, 1H), 7.26-7.30 (m, 1H), 7.41-7.47 (m, 3H), 7.53-7.58 (m, 1H), 7.82 (m, 2H), 8.56 (d, J=0.9 Hz, 1H).

### Example 7

### 4-Benzofuran-2-yl-1H-pyrrolo[2,3-c]pyridin-5-ylamine

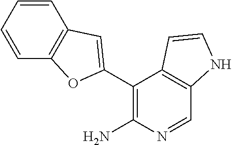

To a degassed mixture of dioxane (5 mL) and water (1 mL) was added 2-benzofuranboronic acid (114 mg, 0.7 mmol), Pd(dppf)Cl₂.CH₂Cl₂ (19 mg, 0.023 mmol), K₂CO₃ (71 mg, 0.52 mmol) and 4-bromo-1H-pyrrolo[2,3-c]pyridin-5-ylamin (Intermediate K, 100 mg, 0.47 mmol). The reaction mixture was heated under reflux for 12 h and cooled to room temperature. It was then filtered through glass filter paper, the filtrate was concentrated and the residue was heated with ethyl acetate and cooled to give 38 mg (33%) of pure target product. ¹H NMR (300 MHz, CDCl₃): δ 5.55 (bs, 2H), 6.71 (m, 1H), 7.25-7.34 (m, 3H), 7.53-7.55 (m, 1H), 7.64 (m, 2H), 8.30 (s, 1H), 11.33 (bs, 1H).

### Example 8

### [(4-Benzofuran-2-yl)-1-benzyl-1H-pyrrolo[2,3-c]pyridin-5-yl]amine

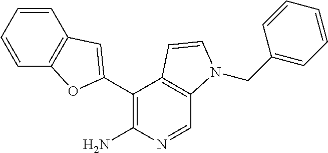

A mixture of 1-benzyl-4-bromo-1H-pyrrolo[2,3-c]pyridin-5-ylamine (Intermediate M, 22 mg, 0.078 mmol), Pd(dppf)Cl₂ (2 mg, 0.015 mmol), 2-benzofuranyl boronic acid (15 mg, 0.09 mmol) and potassium carbonate (13 mg, 0.09 mmol) in degassed dioxane (4 mL) and water (1 mL) was heated under reflux for 12 h. The crude reaction mixture was filtered through glass filter paper and the filtrate was concentrated to give a residue, which was purified by column chromatography (SiO₂, 95% DCM/MeOH) to give 12 mg (48%) of the title compound. ¹H NMR (300 MHz, CDCl₃): δ 5.01 (bs, 2H), 5.34 (s, 2H), 6.78-6.79 (m, 1H), 7.11-7.12 (m, 1H), 7.15-7.17 (m, 2H), 7.27-7.33 (m, 6H), 7.55-7.65 (m, 2H), 8.23 (s, 1H).

### Example 9

### tert-Butyl 4-{2-[5-amino-(4-benzofuran-2-yl)pyrrolo[2,3-c]pyridin-1-yl]ethyl}piperidine-1-carboxamide

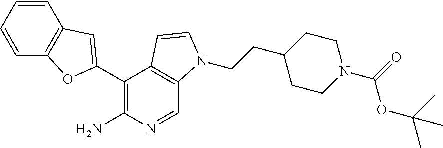

A mixture of tert-butyl 4-[2-(5-amino-4-bromopyrrolo[2,3-c]pyridin-1-yl)ethyl]piperidine-1-carboxamide (Intermediate N, 32 mg, 0.078 mmol), Pd(dppf)Cl₂ (3 mg, 0.015 mmol), 2-benzofuranyl boronic acid (15 mg, 0.09 mmol) and potassium carbonate (15 mg, 0.12 mmol) in degassed dioxane (4 mL) and water (1 mL) was heated under reflux for 12 h. The crude reaction mixture was filtered through glass filter paper and the filtrate was concentrated to give a residue, which was purified by column chromatography (SiO₂, 95% DCM/MeOH) to afford 6 mg (17%) of the title compound. ¹H NMR (300 MHz, CDCl₃): δ 1.43 (s, 9H), 1.70-1.83 (5H), 2.70 (3H), 4.01-4.08 (m, 3H), 4.26-4.30 (m, 2H), 6.73 (m, 1H), 7.18 (d, J=6 Hz, 1H), 7.25-7.33 (m, 2H), 7.47-7.49 (m, 1H), 7.55-7.66 (m, 2H), 8.31 (s, 1H).

### Example 10

### [(4-Benzofuran-2-yl)-1-(2-piperidin-4-ylethyl)-1H-pyrrolo[2,3-c]pyridin-5-yl]amine bis-hydrochloride

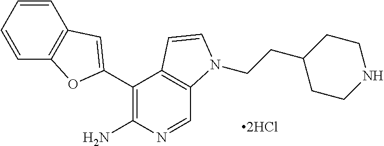

To a solution of tert-butyl 4-{2-[5-amino-(4-benzofuran-2-yl)pyrrolo[2,3-c]pyridin-1-yl]ethyl}piperidine-1-carboxamide (3 mg, 0.006 mmol) in DCM (1 mL) was added 4M HCl in dioxane (1 drop). The reaction mixture was stirred at room temperature for 15 h, concentrated to give 2.5 mg (100%) of the desired product.

### Biological Properties

In some aspects, compounds of the invention are useful as inhibitors of kinases, including one or more of AXL, Tie-2, Flt3, FGFR3, Abl, Aurora A, Aurora B, Jak2, c-Src, IGF-1R, PAK1, PAK2, and TAK1 kinases. In some aspects, compounds of the invention are useful as inhibitors of kinases, including one or more of the above and/or Blk, c-Raf, PRK2, Lck, Mek1, PDK-1, GSK3β, EGFR, p70S6K, BMX, SGK, CaMKII, and Tie-2 kinases.

In some aspects, compounds of the invention are useful as selective inhibitors of one or more of c-MET and/or RON and/or ALK. In some embodiments, the compound is useful as a selective inhibitor of c-MET and/or RON and/or ALK over other kinase targets.

In some aspects of the invention, compounds of the invention are useful as inhibitors of kinases, including at least one of c-MET or RON.

In some aspects of the invention, compounds of the invention are useful as selective inhibitors of both c-MET and RON. In some embodiments, the compound is a selective inhibitor of c-MET and/or RON over other kinase targets, such as KDR and/or AKB.

In some aspects, a compound of the invention exhibits inhibition of c-MET in a cellular assay with an IC₅₀ of about 50 nM or less, about 100 nM or less, about 200 nM or less, about 500 nM or less, about 1 μM or less, or about 10 μM or less.

In some aspects, a compound of the invention exhibits inhibition of RON in a cellular assay with an IC₅₀ of about 50 nM or less, about 100 nM or less, about 200 nM or less, about 500 nM or less, about 1 μM or less, or about 10 μM or less.

In some aspects, a compound of the invention exhibits inhibition of c-MET in a cellular assay with an IC₅₀ selected from above and inhibition of RON in a cellular assay with an IC₅₀ selected from above.

In some aspects, a compound of the invention exhibits inhibition of c-Met in a cellular assay with an IC₅₀ selected from above and inhibition of Ron in a cellular assay with an IC₅₀ selected from above, and which is at least about 10-fold selective for c-Met an/dor RON over KDR.

Compounds of the invention inhibit the activity of tyrosine kinase enzymes in animals, including humans, and may be useful in the treatment and/or prevention of various diseases and conditions. In particular, compounds disclosed herein are inhibitors of such kinases, in particular, but not limited to the above and can be used in the treatment of proliferative diseases, such as, but not limited to, cancer. Compounds disclosed herein may also be useful in the treatment and/or prevention of various diseases and conditions in which EMT is involved, for example, the treatment of conditions characterized by a disregulation of EMT.

The following assays and their respective methods can be carried out with the compounds according to the invention. Activity possessed by compounds of Formula I may be further demonstrated in vivo.

RON (human)-Kₘ of ATP: RON assay is performed in a 384 well assay containing 200 ng/μL biotinylated poly(Glu, Tyr), 0.334 mM vanadate, desired concentration of ATP optimized for the enzyme in assay buffer (50 mM HEPES (pH=7.4), 12.5 mM MgCl₂ and 1% glycerol). Desired compound is added in a final concentration of 1% DMSO with control being vehicle of DMSO alone. RON is diluted to the optimized (on a lot-by-lot basis) concentration in an enzyme diluent buffer (50 mM HEPES pH=7.4, 12.5 mM MgCl₂ and 1% glycerol, 0.03% Brij35, 0.3 mM EGTA, 1 mM DTT, and 0.003% BSA). Enzyme is added to initiate the reaction and incubated for 30 min at RT. In subdued light, appropriate amount of PT66 donor and acceptor beads (diluted 1:260 from manufacturer's provision in a 25 mM Tris HCl (pH=7.5), 200 mM NaCl, 100 mM EDTA, 0.3% BSA buffer) are added to the wells. The plates, incubated for 1 h, are read on an AlphaQuest plate reader.

MET (human)-Km of ATP: MET assay is performed in a 384 well assay containing 200 ng/μL biotinylated poly(Glu, Tyr), 0.334 mM vanadate, desired concentration of ATP optimized for the enzyme in assay buffer (50 mM HEPES (pH=7.4), 5 mM MgCl₂, 5 mM MnCl₂, and 1% glycerol). Desired compound is added in a final concentration of 1% DMSO with control being vehicle of DMSO alone. MET is diluted to the optimized concentration (optimized on a lot-by-lot basis) in an enzyme diluent buffer (50 mM Tris pH=7.4, 1% glycerol, 0.03% Brij35, 0.24 mM EGTA, 1 mM DTT, and 0.003% BSA). Enzyme is added to initiate the reaction and incubated for 60 min at RT. In subdued light, appropriate amount of PT66 donor and acceptor beads (diluted 1:260 from manufacturer's provision in a 25 mM Tris HCl (pH=7.5), 400 mM NaCl, 100 mM EDTA, 0.3% BSA buffer) are added to the wells. The plates, incubated for 1 h, are read on an AlphaQuest plate reader

Activities of exemplary compounds of the present invention are shown in Table 1. Biochemical activities of exemplary compounds of the present invention determined using the assays described herein are shown in Table 1: A, IC₅₀≦5 μM; B, 5 μM<IC₅₀≦20 μM; C, IC₅₀>20 μM; NT, not tested.

TABLE 1

| Example | RON | MET |
|---|---|---|
| 1 | B | B |
| 2 | C | B |
| 3 | NT | NT |
| 4 | NT | NT |
| 5 | A | A |
| 6 | C | B |
| 7 | C | C |
| 8 | C | NT |
| 9 | NT | NT |
| 10 | A | A |

### Compositions

The invention includes pharmaceutical compositions comprising a compound or pharmaceutically acceptable salt thereof of the invention, which is formulated for a desired mode of administration with or without one or more pharmaceutically acceptable and useful carriers. The compounds can also be included in pharmaceutical compositions in combination with one or more other therapeutically active compounds.

The pharmaceutical compositions of the present invention comprise a compound of the invention (or a pharmaceutically acceptable salt thereof) as an active ingredient, optional pharmaceutically acceptable carrier(s) and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Compounds of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

### Uses

In some aspects, the invention provides method of treating a patient having a condition which is mediated by protein kinase activity, such as one or more of the kinases referred to herein, said method comprising administering to the patient a therapeutically effective amount of a compound or salt of any one of the invention. In some aspects, the invention includes a method of treating a condition mediated by protein kinase activity, such as a hyperproliferative disorder. In some aspects, the condition mediated by protein kinase activity is cancer.

In some aspects, compounds of the invention are useful as inhibitors of kinases, including one or more of AXL, Tie-2, Flt3, FGFR3, Abl, Aurora A, Aurora B, Jak2, c-Src, IGF-1R, PAK1, PAK2, and TAK1 kinases. In some aspects, compounds of the invention are inhibitors of kinases, including one or more of the above and/or one or more of Blk, c-Raf, PRK2, Lck, Mek1, PDK-1, GSK36, EGFR, p70S6K, BMX, SGK, CaMKII, and Tie-2 kinases.

In some aspects, compounds of the invention are useful as inhibitors of kinases, including at least one of the c-MET, ALK, and RON kinases.

In some aspects, compounds of the invention are useful as selective inhibitors of one or more of c-MET and/or RON and/or ALK. In some embodiments, the compound is useful as a selective inhibitor of c-MET and/or RON and/or ALK over other kinase targets, such as KDR and/or Aurora kinase B (AKB). In some aspects, compounds of the invention are useful as selective inhibitors of one or more of c-MET, RON, and ALK with selectivity over Aurora kinase B (AKB). In some aspects, compounds of the invention are useful as selective inhibitors of one or more of c-MET, RON, and ALK with selectivity over KDR and/or AKB of 2, 4, 8, 16, or 32-fold, or greater.

In some aspects, the invention includes a method of treating cancer, tumors, and tumor metastases, comprising administering to a mammal in need thereof a therapeutically effective amount of a compound or salt of the invention.

In some aspects, compounds of the invention are in particular useful in treating proliferative disease, particularly cancers, including cancers mediated by c-MET and/or RON and/or ALK, alone or in combination with other agents.

The compounds of Formula I of the present invention are useful in the treatment of a variety of cancers, including, but not limited to, solid tumor, sarcoma, fibrosarcoma, osteoma, melanoma, retinoblastoma, rhabdomyosarcoma, glioblastoma, neuroblastoma, teratocarcinoma, hematopoietic malignancy, and malignant ascites. More specifically, the cancers include, but not limited to, lung cancer, bladder cancer, pancreatic cancer, kidney cancer, gastric cancer, breast cancer, colon cancer, prostate cancer (including bone metastases), hepatocellular carcinoma, ovarian cancer, esophageal squamous cell carcinoma, melanoma, an anaplastic large cell lymphoma, an inflammatory myofibroblastic tumor, and a glioblastoma.

In some aspects, the above methods are used to treat one or more of bladder, colorectal, nonsmall cell lung, breast, or pancreatic cancer. In some aspects, the above methods are used to treat one or more of ovarian, gastric, head and neck, prostate, hepatocellular, renal, glioma, glioma, or sarcoma cancer.

In some aspects, the invention includes a method, including the above methods, wherein the compound is used to inhibit EMT.

In some aspects, the invention includes a method of treating cancer comprising administering to a mammal in need thereof a therapeutically effective amount of a compound or salt of the invention, wherein at least one additional active anti-cancer agent is used as part of the method. In some aspects, the additional agent(s) is an EGFR inhibitor and/or an IGF-1R inhibitor. The agents can be administered together or sequentially according to appropriate considerations such as PK/PD and toxicity. In some aspects, the additional agent(s) is synergistic with the compound of the invention. In some embodiments, the additional agent(s) is one directed to a target(s) for which there is cross-talk with RON and/or c-MET. In some embodiments, the additional agent is an IGF-1R or EGFR inhibitor.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

### GENERAL DEFINITIONS AND ABBREVIATIONS

Except where otherwise indicated, the following general conventions and definitions apply. Unless otherwise indicated herein, language and terms are to be given their broadest reasonable interpretation as understood by the skilled artisan. Any examples given are nonlimiting.

Any section headings or subheadings herein are for the reader's convenience and/or formal compliance and are non-limiting.

A recitation of a compound herein is open to and embraces any material or composition containing the recited compound (e.g., a composition containing a racemic mixture, tautomers, epimers, stereoisomers, impure mixtures, etc.). In that a salt, solvate, or hydrate, polymorph, or other complex of a compound includes the compound itself, a recitation of a compound embraces materials containing such forms. Isotopically labeled compounds are also encompassed except where specifically excluded. For example, hydrogen is not limited to hydrogen containing zero neutrons.

The term “active agent” of the invention means a compound of the invention in any salt, polymorph, crystal, solvate, or hydrated form.

The term “pharmaceutically acceptable salt(s)” is known in the art and includes salts of acidic or basic groups which can be present in the compounds and prepared or resulting from pharmaceutically acceptable bases or acids.

The term “substituted” and substitutions contained in formulas herein refer to the replacement of one or more hydrogen radicals in a given structure with a specified radical, or, if not specified, to the replacement with any chemically feasible radical. When more than one position in a given structure can be substituted with more than one substituent selected from specified groups, the substituents can be either the same or different at every position (independently selected) unless otherwise indicated. In some cases, two positions in a given structure can be substituted with one shared substituent. It is understood that chemically impossible or highly unstable configurations are not desired or intended, as the skilled artisan would appreciate.

In descriptions and claims where subject matter (e.g., substitution at a given molecular position) is recited as being selected from a group of possibilities, the recitation is specifically intended to include any subset of the recited group. In the case of multiple variable positions or substituents, any combination of group or variable subsets is also contemplated.

Unless indicated otherwise, a substituent, diradical or other group referred to herein can be bonded through any suitable position to a referenced subject molecule. For example, the term “indolyl” includes 1-indolyl, 2-indolyl, 3-indolyl, etc.

The convention for describing the carbon content of certain moieties is “(C_{a-b})” or “Cₐ-C_{b}” meaning that the moiety can contain any number of from “a” to “b” carbon atoms. C₀alkyl means a single covalent chemical bond when it is a connecting moiety, and a hydrogen when it is a terminal moiety. Similarly, “x-y” can indicate a moiety containing from x to y atoms, e.g., ₅₋₆heterocycloalkyl means a heterocycloalkyl having either five or six ring members. “C_{x-y}” may be used to define number of carbons in a group. For example, “C₀₋₁₂alkyl” means alkyl having 0-12 carbons, wherein C₀alkyl means a single covalent chemical bond when a linking group and means hydrogen when a terminal group.

The term “absent,” as used herein to describe a structural variable (e.g., “—R— is absent”) means that diradical R has no atoms, and merely represents a bond between other adjoining atoms, unless otherwise indicated.

Unless otherwise indicated (such as by a connecting “−”), the connections of compound name moieties are at the rightmost recited moiety. That is, the substituent name starts with a terminal moiety, continues with any bridging moieties, and ends with the connecting moiety. For example, “heteroarylthioC₁₋₄alkyl is a heteroaryl group connected through a thio sulfur to a C₁₋₄ alkyl, which alkyl connects to the chemical species bearing the substituent.

The term “aliphatic” means any hydrocarbon moiety, and can contain linear, branched, and cyclic parts, and can be saturated or unsaturated.

The term “alkyl” means any saturated hydrocarbon group that is straight-chain or branched. Examples of alkyl groups include methyl, ethyl, propyl, 2-propyl, n-butyl, iso-butyl, tert-butyl, pentyl, and the like.

The term “alkenyl” means any ethylenically unsaturated straight-chain or branched hydrocarbon group. Representative examples include, but are not limited to, ethenyl, 1-propenyl, 2-propenyl, 1-, 2-, or 3-butenyl, and the like.

The term “alkynyl” means any acetylenically unsaturated straight-chain or branched hydrocarbon group. Representative examples include, but are not limited to, ethynyl, 1-propynyl, 2-propynyl, 1-, 2-, or 3-butynyl, and the like.

The term “alkoxy” means —O-alkyl, —O-alkenyl, or —O-alkynyl. “Haloalkoxy” means an —O-(haloalkyl) group. Representative examples include, but are not limited to, trifluoromethoxy, tribromomethoxy, and the like.

“Haloalkyl” means an alkyl, preferably lower alkyl, that is substituted with one or more same or different halo atoms.

“Hydroxyalkyl” means an alkyl, preferably lower alkyl, that is substituted with one, two, or three hydroxy groups; e.g., hydroxymethyl, 1 or 2-hydroxyethyl, 1,2-, 1,3-, or 2,3-dihydroxypropyl, and the like.

The term “alkanoyl” means —C(O)-alkyl, —C(O)-alkenyl, or —C(O)-alkynyl.

“Alkylthio” means an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

The term “cyclic” means any ring system with or without heteroatoms (N, O, or S(O)₀₋₂), and which can be saturated or unsaturated. Ring systems can be bridged and can include fused rings. The size of ring systems may be described using terminology such as “_{x-y}cyclic,” which means a cyclic ring system that can have from x to y ring atoms. For example, the term “₉₋₁₀carbocyclic” means a 5, 6 or 6,6 fused bicyclic carbocyclic ring system which can be satd., unsatd. or aromatic. It also means a phenyl fused to one 5 or 6 membered satd. or unsatd. carbocyclic group. Nonlimiting examples of such groups include naphthyl, 1,2,3,4 tetrahydronaphthyl, indenyl, indanyl, and the like.

The term “carbocyclic” means a cyclic ring moiety containing only carbon atoms in the ring(s) without regard to aromaticity. A 3-10 membered carbocyclic means chemically feasible monocyclic and fused bicyclic carbocyclics having from 3 to 10 ring atoms. Similarly, a 4-6 membered carbocyclic means monocyclic carbocyclic ring moieties having 4 to 6 ring carbons, and a 9-10 membered carbocyclic means fused bicyclic carbocyclic ring moieties having 9 to 10 ring carbons.

The term “cycloalkyl” means a non-aromatic 3-12 carbon mono-cyclic, bicyclic, or polycyclic aliphatic ring moiety. Cycloalkyl can be bicycloalkyl, polycycloalkyl, bridged, or spiroalkyl. One or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like.

The term “unsaturated carbocyclic” means any cycloalkyl containing at least one double or triple bond. The term “cycloalkenyl” means a cycloalkyl having at least one double bond in the ring moiety.

The terms “bicycloalkyl” and “polycycloalkyl” mean a structure consisting of two or more cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1]heptyl (“norbornyl”), bicyclo[2.2.2]octyl, and the like.

The term “spiroalkyl” means a structure consisting of two cycloalkyl moieties that have exactly one atom in common. Examples include, but are not limited to, spiro[4.5]decyl, spiro[2.3]hexyl, and the like.

The term “aromatic” means a planar ring moieties containing 4n+2 pi electrons, wherein n is an integer.

The term “aryl” means an aromatic moieties containing only carbon atoms in its ring system. Non-limiting examples include phenyl, naphthyl, and anthracenyl. The terms “aryl-alkyl” or “arylalkyl” or “aralkyl” refer to any alkyl that forms a bridging portion with a terminal aryl.

“Aralkyl” means alkyl, preferably lower alkyl, that is substituted with an aryl group as defined above; e.g., —CH₂ phenyl, —(CH₂)₂ phenyl, —(CH₂)₃ phenyl, —CH₃CH(CH₃)CH₂phenyl, and the like and derivatives thereof.

The term “heterocyclic” means a cyclic ring moiety containing at least one heteroatom (N, O, or S(O)₀₋₂), including heteroaryl, heterocycloalkyl, including unsaturated heterocyclic rings.

The term “heterocycloalkyl” means a non-aromatic monocyclic, bicyclic, or polycyclic heterocyclic ring moiety of 3 to 12 ring atoms containing at least one ring having one or more heteroatoms. The rings may also have one or more double bonds. However, the rings do not have a completely conjugated pi-electron system. Examples of heterocycloalkyl rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocycloalkyl rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocycloalkyl rings. The term “heterocycloalkyl” also includes fused ring systems and can include a carbocyclic ring that is partially or fully unsaturated, such as a benzene ring, to form benzofused heterocycloalkyl rings. For example, 3,4-dihydro-1,4-benzodioxine, tetrahydroquinoline, tetrahydroisoquinoline and the like. The term “heterocycloalkyl” also includes heterobicycloalkyl, heteropolycycloalkyl, or heterospiroalkyl, which are bicycloalkyl, polycycloalkyl, or spiroalkyl, in which one or more carbon atom(s) are replaced by one or more heteroatoms selected from O, N, and S. For example, 2-oxa-spiro[3.3]heptane, 2,7-diaza-spiro[4.5]decane, 6-oxa-2-thia-spiro[3.4]octane, octahydropyrrolo[1,2-a]pyrazine, 7-aza-bicyclo[2.2.1]heptane, 2-oxa-bicyclo[2.2.2]octane, and the like, are such heterocycloalkyls.

Examples of saturated heterocyclic groups include, but are not limited to oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl, 1,4-diazepanyl

Non-aryl heterocyclic groups include satd. and unsatd. systems and can include groups having only 4 atoms in their ring system. The heterocyclic groups include benzo-fused ring systems and ring systems substituted with one or more oxo moieties. Recitation of ring sulfur is understood to include the sulfide, sulfoxide or sulfone where feasible. The heterocyclic groups also include partially unsatd. or fully satd. 4-10 membered ring systems, e.g., single rings of 4 to 8 atoms in size and bicyclic ring systems, including aromatic 6-membered aryl or heteroaryl rings fused to a non-aromatic ring. Also included are 4-6 membered ring systems (“4-6 membered heterocyclic”), which include 5-6 membered heteroaryls, and include groups such as azetidinyl and piperidinyl. Heterocyclics can be heteroatom-attached where such is possible. For instance, a group derived from pyrrole can be pyrrol-1-yl (N-attached) or pyrrol-3-yl (C-attached). Other heterocyclics include imidazo[4,5-b]pyridin-3-yl and benzoimidazol-1-yl.

Examples of heterocyclic groups include pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, and the like.

The term “unsaturated heterocyclic” means a heterocycloalkyl containing at least one unsaturated bond. The term “heterobicycloalkyl” means a bicycloalkyl structure in which at least one carbon atom is replaced with a heteroatom. The term “heterospiroalkyl” means a spiroalkyl structure in which at least one carbon atom is replaced with a heteroatom.

Examples of partially unsaturated heteroalicyclic groups include, but are not limited to 3,4-dihydro-2H-pyranyl, 5,6-dihydro-2H-pyranyl, 2H-pyranyl, 1,2,3,4-tetrahydropyridinyl, and 1,2,5,6-tetrahydropyridinyl.

The terms “heteroaryl” or “hetaryl” mean a monocyclic, bicyclic, or polycyclic aromatic heterocyclic ring moiety containing 5-12 atoms. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, and the like. Furthermore, the terms “heteroaryl” include fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

Heteroaryls include, e.g., 5 and 6 membered monocyclics such as pyrazinyl and pyridinyl, and 9 and 10 membered fused bicyclic ring moieties, such as quinolinyl. Other examples of heteroaryl include quinolin-4-yl, 7-methoxy-quinolin-4-yl, pyridin-4-yl, pyridin-3-yl, and pyridin-2-yl. Other examples of heteroaryl include pyridinyl, imidazolyl, pyrimidinyl, pyrazolyl, triazolyl, pyrazinyl, tetrazolyl, furanyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. Examples of 5-6 membered heteroaryls include, thiophenyl, isoxazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl, 1,3,5-triazinyl, and the like.

“Heteroaralkyl” group means alkyl, preferably lower alkyl, that is substituted with a heteroaryl group; e.g., —CH₂ pyridinyl, —(CH₂)₂pyrimidinyl, —(CH₂)₃imidazolyl, and the like, and derivatives thereof.

A pharmaceutically acceptable heteroaryl is one that is sufficiently stable to be attached to a compound of the invention, formulated into a pharmaceutical composition and subsequently administered to a patient in need thereof.

Examples of monocyclic heteroaryl groups include, but are not limited to: pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl.

Examples of fused ring heteroaryl groups include, but are not limited to: benzoduranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, azaquinazoline, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrimido[2,3-b]pyrazinyl, pyrimido[4,5-d]pyrimidinyl.

“Arylthio” means an —S-aryl or an —S-heteroaryl group, as defined herein. Representative examples include, but are not limited to, phenylthio, pyridinylthio, furanylthio, thienylthio, pyrimidinylthio, and the like and derivatives thereof.

The term “9-10 membered heterocyclic” means a fused 5, 6 or 6,6 bicyclic heterocyclic ring moiety, which can be satd., unsatd. or aromatic. The term “9-10 membered fused bicyclic heterocyclic” also means a phenyl fused to one 5 or 6 membered heterocyclic group. Examples include benzofuranyl, benzothiophenyl, indolyl, benzoxazolyl, 3H-imidazo[4,5-c]pyridin-yl, dihydrophthazinyl, 1H-imidazo[4,5-c]pyridin-1-yl, imidazo[4,5-b]pyridyl, 1,3 benzo[1,3]dioxolyl, 2H-chromanyl, isochromanyl, 5-oxo-2,3 dihydro-5H-[1,3]thiazolo[3,2-a]pyrimidyl, 1,3-benzothiazolyl, 1,4,5,6 tetrahydropyridinyl, 1,2,3,4,7,8 hexahydropteridinyl, 2-thioxo-2,3,6,9-tetrahydro-1H-purin-8-yl, 3,7-dihydro-1H-purin-8-yl, 3,4-dihydropyrimidin-1-yl, 2,3-dihydro-1,4-benzodioxinyl, benzo[1,3]dioxolyl, 2H-chromenyl, chromanyl, 3,4-dihydrophthalazinyl, 2,3-dihydro-1H-indolyl, 1,3-dihydro-2H-isoindol-2-yl, 2,4,7-trioxo-1,2,3,4,7,8-hexahydropteridin-yl, thieno[3,2-d]pyrimidinyl, 4-oxo-4,7-dihydro-3H-pyrrolo[2,3-d]pyrimidin-yl, 1,3-dimethyl-6-oxo-2-thioxo-2,3,6,9-tetrahydro-1H-purinyl, 1,2-dihydroisoquinolinyl, 2-oxo-1,3-benzoxazolyl, 2,3-dihydro-5H-1,3-thiazolo-[3,2-a]pyrimidinyl, 5,6,7,8-tetrahydro-quinazolinyl, 4-oxochromanyl, 1,3-benzothiazolyl, benzimidazolyl, benzotriazolyl, purinyl, furylpyridyl, thiophenylpyrimidyl, thiophenylpyridyl, pyrrolylpiridyl, oxazolylpyridyl, thiazolylpiridyl, 3,4-dihydropyrimidin-1-yl imidazolylpyridyl, quinoliyl, isoquinolinyl, quinazolinyl, quinoxalinyl, naphthyridinyl, pyrazolyl[3,4]pyridine, 1,2-dihydroisoquinolinyl, cinnolinyl, 2,3-dihydro-benzo[1,4]dioxin-4-yl, 4,5,6,7-tetrahydro-benzo[b]-thiophenyl-2-yl, 1,8-naphthyridinyl, 1,5-napthyridinyl, 1,6-naphthyridinyl, 1,7-napthyridinyl, 3,4-dihydro-2H-1,4-benzothiazine, 4,8-dihydroxy-quinolinyl, 1-oxo-1,2-dihydro-isoquinolinyl, 4-phenyl-[1,2,3]thiadiazolyl, and the like.

“Aryloxy” means an —O-aryl or an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

One in the art understands that an “oxo” requires a second bond from the atom to which the oxo is attached. Accordingly, it is understood that oxo cannot be subststituted onto an aryl or heteroaryl ring.

The term “halo” or “halogen” means fluoro, chloro, bromo, or iodo.

“Acyl” means a —C(O)R group, where R can be selected from the nonlimiting group of hydrogen or optionally substituted lower alkyl, trihalomethyl, unsubstituted cycloalkyl, aryl. “Thioacyl” or “thiocarbonyl” means a—C(S)R group, with R as defined above.

The term “carboxy” means a —C(O)OR group, where R can be H or an optional substituent.

The term “amido” means —C(O)NR^{a}R^{b} or —NR^{a}C(O)R^{b}, wherein R^{a} and R^{b} can be H or an optional substituent.

The terms sulfide, sulfoxide, and sulfone together mean —S(O)₀₋₂R, where R is an optionally substituted carbon atom.

The term “protecting group” means a suitable chemical group that can be attached to a functional group and removed at a later stage to reveal the intact functional group. Examples of suitable protecting groups for various functional groups are described in T. W. Greene and P. G. M. Wuts, Protective Groups in Organic Synthesis, 2d Ed., John Wiley and Sons (1991 and later editions); L. Fieser and M. Fieser, Fieser and Fieser's Reagents for Organic Synthesis, John Wiley and Sons (1994); and L. Paquette, ed. Encyclopedia of Reagents for Organic Synthesis, John Wiley and Sons (1995). The term “hydroxy protecting group”, as used herein, unless otherwise indicated, includes Ac, CBZ, and various hydroxy protecting groups familiar to those skilled in the art including the groups referred to in Greene.

As used herein, the term “pharmaceutically acceptable salt” means those salts which retain the biological effectiveness and properties of the parent compound and do not present insurmountable safety or toxicity issues.

The term “pharmaceutical composition” means an active compound in any form suitable for effective administration to a subject, e.g., a mixture of the compound and at least one pharmaceutically acceptable carrier.

As used herein, a “physiologically/pharmaceutically acceptable carrier” means a carrier or diluent that does not cause significant irritation to an organism and does not abrogate the biological activity and properties of the administered compound.

A “pharmaceutically acceptable excipient” means an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The terms “treat,” “treatment,” and “treating” means reversing, alleviating, inhibiting the progress of, or partially or completely preventing the disorder or condition to which such term applies, or one or more symptoms of such disorder or condition. “Preventing” means treating before an infection occurs.

“Therapeutically effective amount” means that amount of the compound being administered which will relieve to some extent one or more of the symptoms of the disorder being treated, or result in inhibition of the progress or at least partial reversal of the condition.

The following abbreviations are used:

min. minute(s)

h hour(s)

d day(s)

RT or rt room temperature

t_{R} retention time

L liter

mL milliliter

mmol millimole

μmol micromole

equiv. or eq. equivalents

NMR nuclear magnetic resonance

MDP(S) mass-directed HPLC purification (system)

LC/MS liquid chromatography mass spectrometry

HPLC high performance liquid chromatography

TLC thin layer chromatography

CDCl₃ deuterated chloroform

CD₃OD or MeOD deuterated methanol

DMSO-d₆ deuterated dimethylsulfoxide

LDA lithium diisopropylamide

DCM dichloromethane

THF tetrahydrofuran

EtOAc ethyl acetate

MeCN acetonitrile

DMSO dimethylsulfoxide

Boc tert-butyloxycarbonyl

DME 1,2-dimethoxyethane

DMF N,N-dimethylformamide

DIPEA diisopropylethylamine

PS-DIEA polymer-supported diisopropylethylamine

PS-PPh₃-Pd polymer-supported Pd(PPh₃)₄

EDC 1-(3-dimethylaminopropyl)-3-ethylcarbodiimide

HOBt 1-hydroxybenzotriazole

DMAP 4-dimethylaminopyridine

TBTU O-(benzotriazol-1-yl)-N,N,N′,N′-tetramethyluroniurn tetrafluoroborate

TEMPO 2,2,6,6-tetramethylpiperidine-1-oxyl

TFA trifluoroacetic acid

1. A compound of Formula I: 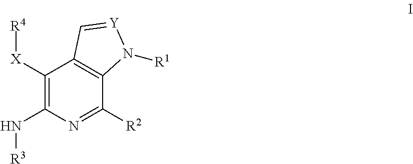
or a pharmaceutically acceptable salt thereof, wherein:
X is O, S(O)₀₋₂, or NR⁵;
or X is absent;
Y is C—R⁶ or N;
R¹ is H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic, heteroarylC₀₋₁₂aliphatic, OR⁷, —S(O)₀₋₂R⁸, —NR⁹R¹⁰, —C(O)R^{a}, —C(O)NR⁹R¹⁰; —C(O)—C(O)NR⁹R¹⁰, —C(O)OR⁷, —C(O)—C(O)OR⁷, —OC(O)R^{b}, —NR⁹C(O)R^{a}, —NR⁹S(O)₂R^{a}, —(CR¹¹R¹²)ₙC(O)R^{a}, —(CR¹¹R¹²)ₙC(O)OR⁷, —(CR¹¹R¹²)ₙC(O)NR⁹R¹⁰, —(CR¹¹R¹²)ₙS(O))₂NR⁹R¹⁰; —(CR¹¹R¹²)ₙNR⁹R¹⁰; —(CR¹¹R¹²)ₙOR⁷, —(CR¹¹R¹²)ₙS(O)₀₋₂R⁸, —NR¹³C(O)NR⁹R¹⁰; —NR¹³S(O)₂NR⁹R¹⁰ or —NR¹³S(O)NR⁹R¹⁰, any of which is optionally substituted with one or more independent G¹ substituents;
R² is H or halogen;
R³ is H or C₁₋₁₂aliphatic;
R⁴ is H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic, arylC₃₋₁₂cycloalkyl, arylC₃₋₁₂heterocycloalkyl, heteroarylC₀₋₁₂aliphatic, heteroarylC₃₋₁₂cycloalkyl or heteroarylC₃₋₁₂heterocycloalkyl, any of which is optionally substituted with one or more independent G² substituents;
or R⁴ is —(CR¹⁸R¹⁹)ₙA¹;
A¹ is aryl or heteroaryl optionally substituted by one or more independent G³;
R⁵ is H, C₁₋₁₂aliphatic C₃₋₁₂cycloalkylC₀₋₁₂aliphatic, C₃₋₁₂heterocycloalkylC₀₋₁₂aliphatic, arylC₀₋₁₂aliphatic, heteroarylC₀₋₁₂aliphatic, —O—C₂₋₁₂aliphatic, —S(O)₀₋₂—C₂₋₁₂aliphatic, (C₀₋₁₂aliphatic)(C₀₋₁₂aliphatic)N—C₂₋₁₂aliphatic, any of which is optionally substituted with one or more independent G⁴ substituents;
R⁶ is H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic or heteroarylC₀₋₁₂aliphatic, any of which is optionally substituted with one or more independent G⁵ substituents, or R⁶ is halo, —CN, or —CF₃;
R⁷, R⁸, R⁹, R¹⁰, R¹¹, R¹², R¹³, R¹⁵, R¹⁶, R¹⁷, R¹⁸, R¹⁹, R^{a}, and R_{b} are each independently selected from H, C₁₋₁₂aliphatic, C₃₋₁₂cycloalkylC₀₋₁₂aliphatic, C₃₋₁₂heterocycloalkylC₀₋₁₂aliphatic, arylC₀₋₁₂aliphatic or heteroarylC₀₋₁₂aliphatic;
R⁹ and R¹⁰, or R¹⁶ and R¹⁷ in NR⁹R¹⁰ and NR¹⁶R¹⁷, respectively, can be taken together with the nitrogen atom to which they are attached to form a 3-12 membered saturated or unsaturated ring, wherein said ring optionally includes one or more heteroatoms selected from O, N, or S(O)₀₋₂;
R¹¹ and R¹², or R¹⁸ and R¹⁹ in CR¹¹R¹² and CR¹⁸R¹⁹, respectively, can be taken together with the carbon atom to which they are attached to form a 3-12 membered saturated or unsaturated ring, wherein said ring optionally includes one or more heteroatoms selected from O, N, or S(O)₀₋₂;
G¹, G², G³, G⁴, and G⁵ are each independently selected from H, C₁₋₁₂aliphatic, C₃₋₁₂bycloalkylC₀₋₁₂aliphatic, C₃₋₁₂heterocycloalkylC₀₋₁₂aliphatic, arylC₀₋₁₂aliphatic, arylC₃₋₁₂cycloalkyl, arylC₃₋₁₂heterocycloalkyl, heteroarylC₀₋₁₂aliphatic, heteroarylC₃₋₁₂cycloalkyl or heteroarylC₃₋₁₂heterocycloalkyl, any of which is optionally substituted with one or more independent Q¹ substituents, or G¹, G², G³, G⁴, and G⁵ are each independently halo, —CN, —CF₃, —OCF₃, or —NO₂;
each Q¹ is independently selected from H, C₁₋₁₂aliphatic, C₃₋₁₂cycloalkyl, C₃₋₁₂heterocycloalkyl, aryl, heteroaryl, —C(O)—C(O)NR²⁰R²¹, —C(O)—C(O)OR²², —OC(O)R^{c}, —NR²⁰C(O)R^{c}, —NR²⁰S(O)₂R²³, —(CR²⁴R²⁵)ₙC(O)R^{c}, —(CR²⁴R²⁵)ₙC(O)OR²², —(CR²⁴R²⁵)ₙC(O)NR²⁹R²¹, (CR²⁴R²⁵)ₙS(O)₂NR²⁹R²¹, (CR²⁴R²⁵)ₙNR²⁹R²¹, (CR²⁴R²⁵)ₙOR²², —(CR²⁴R²⁵)ₙS(O)₀₋₂R²³, —NR²⁶C(O)NR²⁹R²¹, NR²⁶S(O)₂NR²⁹R²¹ or —NR²⁶S(O)NR²⁹R²¹, any of which is optionally substituted with one or more independent Q² substituents or Q¹ is halo, —CN, —NO₂, oxo, —CF₃, or —OCF₃;
each Q² is independently selected from H, halo, —CN, —OH, —NH₂, —NO₂, oxo, —CF₃, —OCF₃, —CO₂H, —S(O)₀₋₂H, C₁₋₁₂aliphatic, C₃₋₁₂cycloalkyl, C₃₋₁₂heterocycloalkyl, aryl, heteroaryl, any of which is optionally substituted with one or more independent halo, —CN, —OH, —NH₂ or C₁₋₁₀alkyl which may be partially or fully halogenated, or —O—C₁₋₁₀alkyl which may be partially or fully halogenated;
R²⁰, R²¹, R²², R²³, R²⁴, R²⁵, R²⁶, and R^{c} are each independently selected from H, C₁₋₁₂aliphatic, arylC₀₋₁₂aliphatic, heteroarylC₀₋₁₂aliphatic, C₃₋₁₂cycloalkylC₀₋₁₂aliphatic, C₃₋₁₂-heterocycloalkylC₀₋₁₂aliphatic, arylC₃₋₁₂cycloalkyl, heteroarylC₃₋₁₂cycloalkyl, C₃₋₁₂heterocycloalkylC₃₋₁₂cycloalkyl, C₃₋₁₂cycloalkylC₃₋₁₂cycloalkyl, C₁₋₁₂alkylC₃₋₁₂heterocycloalkyl, C₃₋₁₂heterocycloalkylC₃₋₁₂heterocycloalkyl, arylC₃₋₁₂heterocycloalkyl, or heteroarylC₃₋₁₂heterocycloalkyl substituents;
R²⁰ and R²¹ in NR²⁰R²¹ can be taken together with the nitrogen atom to which they are attached to form a 3-12 membered saturated or unsaturated ring, wherein said ring optionally includes one or more heteroatoms selected from O, N, or S(O)₀₋₂;
R²⁴ and R²⁵ in CR²⁴R²⁵ can be taken together with the carbon atom to which they are attached to form a 3-12 membered saturated or unsaturated ring, wherein said ring optionally includes one or more heteroatoms selected from O, N, or S(O)₀₋₂; and
n=0-7.

2. The compound or salt of claim 1, wherein:
X is O;
Y is CH;
R² is H or Cl;
R³ is H; and
R⁴ is —CH(CH₃)-A¹.

3. The compound or salt of claim 1, having the formula:

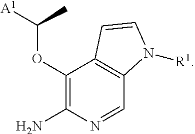

4. The compound or salt of claim 3, wherein: A¹ is phenyl substituted by one or more independent halogen or methoxy optionally substituted by 1-3 fluorine atoms.

5. The compound or salt of claim 3, wherein A¹ is 2,6-dichloro-3-fluorophenyl.

6. The compound or salt of claim 5, wherein R¹ is H, C₁₋₄aliphatic optionally substituted by ₅₋₆cyclic which is optionally substituted, —S(O)₀₋₂R⁸, or —C(O)OR⁷.

7. The compound or salt of claim 5, wherein R¹ is H or C₁₋₄aliphatic optionally substituted by ₅₋₆cyclic which is optionally substituted.

8. The compound or salt of claim 5, wherein R¹ is H or C₁₋₄aliphatic optionally substituted by ₅₋₆heterocyclic which is optionally substituted.

9. The compound or salt of claim 1, having the formula: 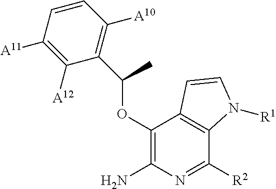
wherein:
A¹⁰ is halogen, methyl, or methoxy either of which is optionally substituted by 1-3 fluorine atoms;
A¹¹ and A¹² are independently halogen;
R¹ is H, C₁₋₄alkyl, —CH₂—CH₂-G¹, —CH₂—CH₂—NH-G¹, or —CH₂—CH₂—O-G¹;
R² is H; and
G¹ is ₄₋₆heterocycloalkyl optionally substituted by one or more substituted or unsubstituted oxo, aliphatic, carboxy, amido, sulfonamido, sulfone, sulfide, sulfoxide, or acyl.

10. The compound or salt of claim 1, having the formula: 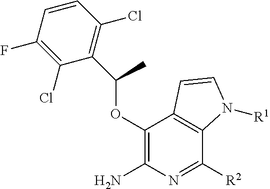
wherein:
R¹ is H or C₁₋₃aliphatic optionally substituted by ₅₋₆cyclic; and
R² is H or halogen.

11. The compound or salt of claim 1, wherein:
X is absent;
Y is CH;
R¹ is H or C₁₋₄alkyl optionally substituted by ₅₋₆cyclic or aryl which is optionally substituted;
R² is H or Cl;
R³ is H; and
R⁴ is benzofuran-2-yl which can be substituted by 1-3 independent halogen, hydroxy, or —OC₀₋₃aliphatic optionally substituted by 1-3 halogen atoms.

12. The compound or salt of claim 11, wherein R² is H.

13. The compound or salt of claim 1, having the formula: 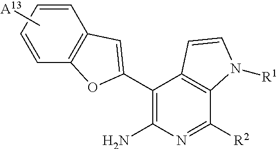
wherein:
A¹³ is H, halogen, methyl, or methoxy either of which is optionally substituted by 1-3 fluorine atoms;
R¹ is H, C₁₋₄alkyl, —CH₂—CH₂-G¹, —CH₂—CH₂—NH-G¹, or —CH₂—CH₂—O-G¹;
R² is H; and
G¹ is ₄₋₆heterocycloalkyl optionally substituted by one or more substituted or unsubstituted oxo, aliphatic, carboxy, amido, sulfonamido, sulfone, sulfide, sulfoxide, or acyl.

14. The compound or salt of claim 1, which exhibits inhibition of RON in a biochemical assay with an IC₅₀ of about 10 mM or less.

15. The compound or salt of claim 1, which exhibits inhibition of MET in a biochemical assay with an IC₅₀ of about 10 mM or less.

16. The compound or salt of any one of the examples herein.

17. A pharmaceutical composition comprising the compound or salt of claim 1, formulated with or without one or more pharmaceutical carriers.

18. A method of treating a cancer mediated at least in part by MET and/or RON comprising administering to a mammal in need thereof a therapeutically effective amount of a compound or salt of claim 1.

19. A method of treating a cancer selected from bladder, colorectal, non-small cell lung, breast, pancreatic, ovarian, gastric, head and neck, prostate, hepatocellular, renal, glioma, or sarcoma cancer comprising administering to a mammal in need thereof a therapeutically effective amount of a compound or salt of claim 1.

20. The method of claim 19, wherein the compound or salt thereof is a dual RON and c-Met inhibitor.

21. (canceled)
